(19) United States
(12) Patent Application Publication
(10) Pub. No.: US 2021/0000386 A1
(43) Pub. Date: Jan. 7, 2021

(54) A SYSTEM AND METHOD FOR DETERMINING POWER AND PROPULSION EFFICIENCY OF A LIMB DURING LIMBED PROPULSION

(71) Applicant: VIBE TECHNOLOGIES LIMITED, Wellington (NZ)

(72) Inventor: Dean Kenneth Whiteman, Wellington (NZ)

(21) Appl. No.: 16/977,296

(22) PCT Filed: Mar. 1, 2019
(86) PCT No.: PCT/NZ2019/050020

§ 371 Date: Sep. 1, 2020

(30) Foreign Application Priority Data
Mar. 2, 2018 (NZ) 740441
Dec. 20, 2018 (NZ) 749498

(51) Int. Cl. A61B 5/11; A61B 5/22; A61B 5/00

(52) U.S. Cl. CPC A61B 5/112; A61B 5/1122; A61B 5/224; A61B 5/6828; A61B 2560/0209; A61B 2503/10; A61B 2562/0219; A61B 5/0004; A61B 5/7221; A61B 5/6807

(57) ABSTRACT

The present invention provides a system for determining power output and propulsion efficiency of a user's limb during limbed propulsion; the system comprising: (a) at least two or more accelerometers to measure the acceleration of the limb in at least two substantially orthogonal planes, (b) a processor means to calculate the power output generated by the limb to derive a limb propulsion efficiency measure, and (c) a transmission means to send the limb power output and limb propulsion efficiency metrics to a receiving device; and wherein the system is wearable on the user's limb.

### FIELD OF THE INVENTION

The present invention relates to a system and a method of determining power output and propulsion efficiency of a limb during limbed propulsion. More specifically, the invention relates to a system and a method of determining running efficiency and running power of a limb, for example the leg, of a human or animal.

### BACKGROUND OF THE INVENTION

Conventional systems for obtaining propulsion or running metrics provide many variables that can be analysed to assist the user, however these metrics are difficult for the average user to understand and generally require expert knowledge to usefully interpret.

Running economy or efficiency is typically defined as the energy demand for a given velocity which is performed at an aerobic pace, and is usually determined by measuring the steady-state consumption of oxygen and the respiratory exchange ratio, to determine energy use. The measured value is typically adjusted based on the mass of the individual. Motion with good running economy/efficiency will use less energy than motion with poor running economy/efficiency at the same velocity.

Several factors have been identified by biomechanists which are known to influence running efficiency or economy/efficiency, such as net vertical impulses of the ground reaction force, vertical leg spring stiffness, stride length and the ground contact time. Other non-biomechanical areas which influence running economy/efficiency include physiological, anthropometrical and psychological factors.

The ground or surface contact time of a user during the Gait cycle is generally acknowledged as a key factor in determining the running economy/efficiency of an individual and several studies including “Factors Related to Top Running Speed and Economy/efficiency, A. Nummela, T. Keränen, L. O. Mikkelsson” have confirmed this as a key metric.

U.S. Pat. No. 7,603,255 describes a “Running Power Parameter” and notes that there is no “power” measurement parameter for running because of the difficulty in accurately measuring power transferred in the shoe. U.S. Pat. No. 7,603,255 then describes a system or method that may use a new derivation of estimate of running power from using a number of data generated from GPS, pressure sensors and/or other sensors carried by the athlete during the event, eg integrating an accelerometer data, determining an approximation for drag and inertia to establish a running power parameter.

Force plates, which are used to measure the ground reaction force as a runner runs over the force plate, can be used to derive running power or running efficiency. These force plates are large plates or pads onto which a runner plants his/her foot during a running action/motion. While force plates can be used to derive running power or running efficiency metrics, force plates are devices that are too large to be worn as a running accessory.

Typically a true measurement of running efficiency or economy is not possible outside of a laboratory setting due to the requirement for gas exchange respiratory equipment to measure energy use, alongside the use of ground reaction force plates and video Gait analysis systems which are generally not portable or able to be used during exercise outside the clinical or laboratory setting.

It is therefore an object of the present invention to provide a device wearable on a limb, and/or a method for determining power output and propulsion efficiency of a limb during limbed propulsion or to at least provide the public with a useful alternative.

### SUMMARY OF THE INVENTION

In a first aspect, the present invention provides a system for determining power output and propulsion efficiency of a user's limb during limbed propulsion; the system comprising:
(a) at least two or more accelerometers to measure the acceleration of the limb in at least two substantially orthogonal planes,
(b) a processor means to calculate the power output generated by the limb to derive a limb propulsion efficiency measure, and
(c) a transmission means to send the limb power output and limb propulsion efficiency metrics to a receiving device; and
   wherein the system is wearable on the user's limb.

In a second aspect, the present invention provides a system for determining power output and propulsion efficiency of a user's limb during limbed propulsion; the system comprising:
(a) at least two or more accelerometers to measure the acceleration of the limb in at least two substantially orthogonal planes,
(b) at least one gyroscope to measure the rotational velocity of the limb;
(c) a processor means to calculate the power output generated by the limb to derive a limb propulsion efficiency measure, and
(d) a transmission means to send the limb power output and limb propulsion efficiency metrics to a receiving device; and

wherein the system is wearable on the user's limb.

In one embodiment the present invention provides a system as defined above having three accelerometers to measure the acceleration of the limb in three substantially orthogonal planes.

In one embodiment the present invention provides a system as defined above for measuring the power output and propulsion efficiency of the user's limb. In one embodiment the limb is a leg.

In one embodiment the present invention provides a system as defined above wherein the two or more accelerometers, in use, measure the limb force transmitted along the user's limb and the limb contact time on the limb contact surface and the system derives the power output and propulsion efficiency of the limb therefrom. In one embodiment the two or more accelerometers measure or are used to derive the velocity of the limb in the horizontal plane relative to the limb contact surface.

In one embodiment the two or more accelerometers and one or more gyroscopes in use measure or are used to derive the velocity of the user's centre of mass in the horizontal plane relative to the limb contact surface.

In one embodiment the two or more accelerometers and one or more gyroscopes in use measure or are used to derive the velocity of the limb in the horizontal plane relative to the limb contact surface.

In a third aspect of the present invention there is provided a method for determining power output and propulsion efficiency of a user's limb during limbed propulsion; the method comprising the steps of:
(a) measuring the acceleration of the limb in at least two substantially orthogonal planes,
(b) calculating the power output generated by the limb from the acceleration measurements to derive a limb propulsion efficiency measure.

In a fourth aspect of the present invention there is provided a method for determining power output and propulsion efficiency of a user's leg whilst running, the method including the steps of:
(a) Measuring the tibial shock/force transmitted along the user's leg as it impacts the ground using a system as defined above;
(b) Deriving a ground reaction force metric from the tibial shock/force measurement and the leg contact time on the ground;
(c) measuring the acceleration in the horizontal plane of the limb or centre of mass using a system as defined above and
(d) Deriving a power output and propulsion efficiency metric therefrom.

In one embodiment the method includes the further step of transmitting the limb or leg power output and limb or leg propulsion efficiency metrics to a receiving device.

It is to be appreciated that the system and methods may be used for different types of users such as humans, animals or robots which use limbs for propulsion.

The foregoing brief summary broadly describes the features and technical advantages of certain embodiments of the present invention.

Novel features that are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to limit the invention's scope.

### BRIEF DESCRIPTION OF THE DRAWINGS

A more complete understanding of the principles of the present invention may be obtained by reference to the drawings in which corresponding reference numbers indicate corresponding parts or features, and wherein:

FIG. 1A shows a running power and running economy/efficiency measurement system, according to some embodiments.

FIG. 113 is a block diagram of a running power and running economy/efficiency measurement device for measuring, calculating and transmitting power and running economy/efficiency metrics to a receiving unit, according to some embodiments.

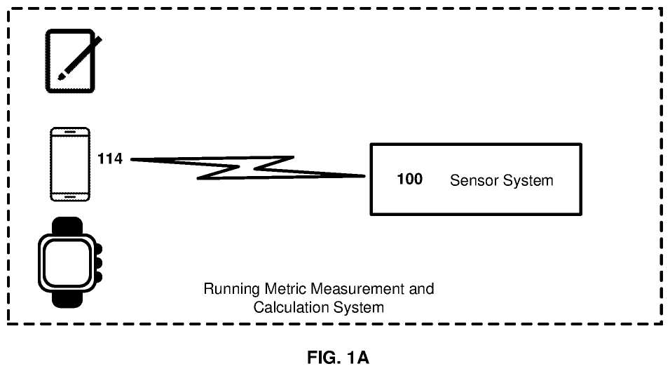

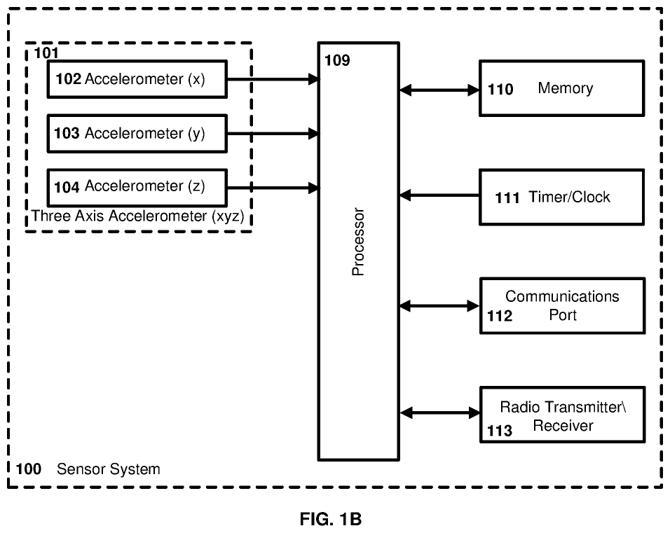

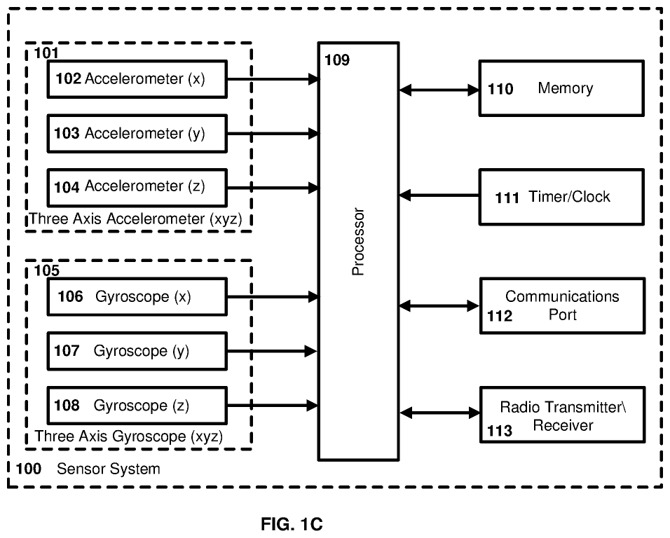

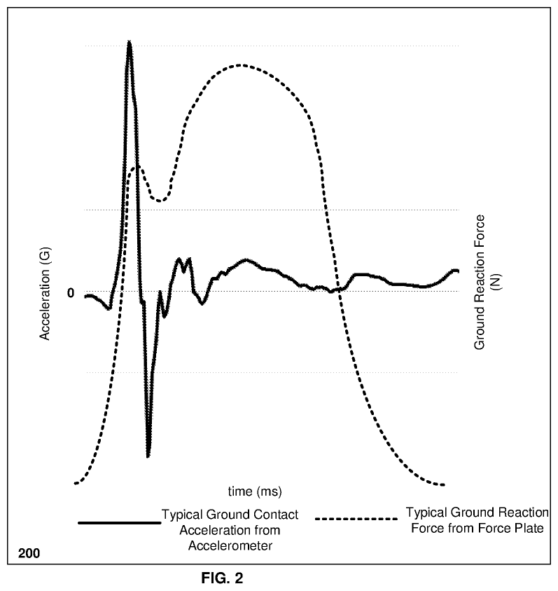

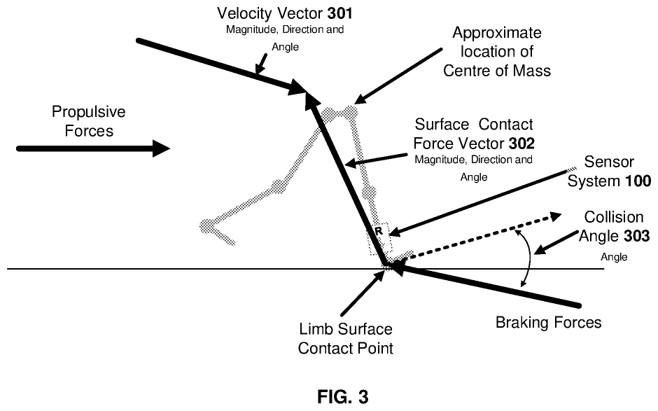

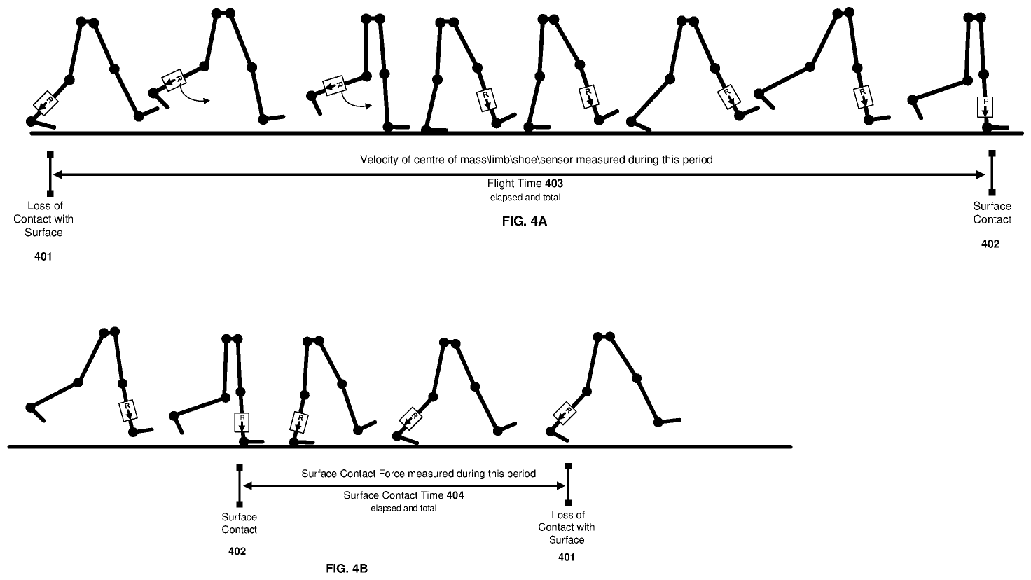

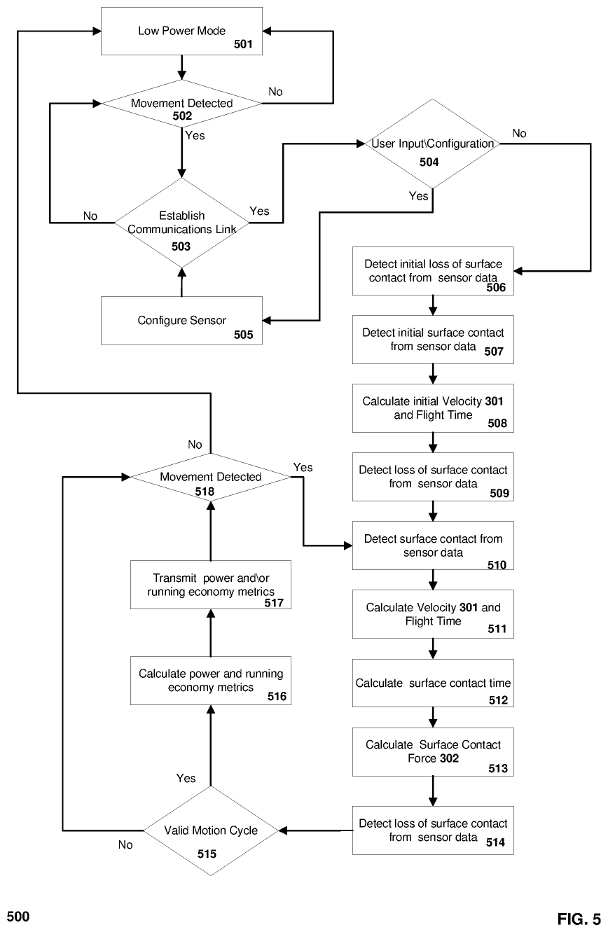

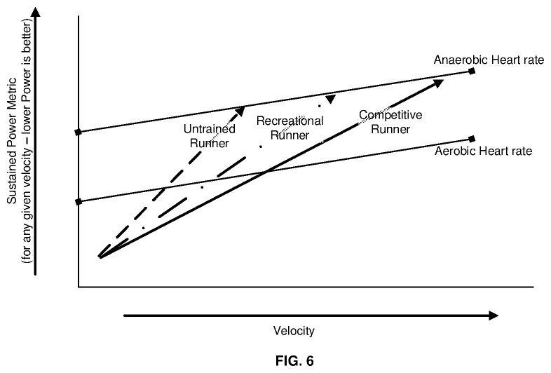

FIG. 1C is a block diagram of a running power and running economy/efficiency measurement device for measuring, calculating and transmitting power and running economy/efficiency metrics to a receiving unit, according to some embodiments.

FIG. 2 is a representation of ground reaction force from a force plate compared with a typical accelerometer waveform captured during the same period.

FIG. 3 illustrates the vectors and forces that can be measured to determine running power and running economy/efficiency, according to the present invention.

FIG. 4A illustrates examples of the points of a typical two limbed Gait cycle which are of interest in determining running economy/efficiency and power, according to some embodiments.

FIG. 4B illustrates examples of the points of a typical two limbed Gait cycle which are of interest in determining running economy/efficiency and power, according to some embodiments.

FIG. 5 is a flow chart illustrating the steps performed by the running power and running economy/efficiency measurement device, according to some embodiments.

FIG. 6 shows a plot that illustrates the relationship between velocity, heart rate, running power and running economy/efficiency in different levels of runner.

### DETAILED DESCRIPTION

The following description sets forth numerous exemplary configurations, parameters, and the like. It should be recognised, however, that such description is not intended as a limitation on the scope of the present invention, but is instead provided as a description of exemplary embodiments.

### Definitions

In each instance herein, in descriptions, embodiments, and examples of the present invention, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

The term “user” as used herein refers to a human, an animal or machine which uses one or more limbs for propulsion or motion.

The term “limb contact time” as used herein refers to the duration of time in which the limb is in contact with the surface against which the limb is being driven to achieve propulsion.

The term “limb contact surface” is the surface against which the limb is brought into contact during the propulsion action.

The terms “propulsion” or “propel” as used herein refer to the act of driving or pushing a user forward, with a limb, and includes for example the use of a limb to walk, crawl, swim, jump, row, kayak, paddle, run and sprint.

The term “power output’ used herein refers to the power generated by a user to propel a limb of the user forward.

The term “propulsion efficiency” used herein refers to the efficiency with which the user is able to generate power to propel the user forward with the limb, and includes for example the propulsion economy/efficiency of the limb(s), such as the running economy or running efficiency.

The terms “tibial shock” or “tibial force” as used interchangeably herein refers to the shock wave that travels up and down the tibia bone (also known as shinbone or shank bone) after the collision of the foot with a ground surface.

The terms “limb shock” or “limb force” as used interchangeably herein refers to the shock wave that travels up and down the limb bone after the collision of the limb with a resisting contact surface.

The present invention relates generally to a system and method for determining both propulsion efficiency, that encompasses running economy, sometimes known as running efficiency, and power output, that encompasses running power using a limb collision based approach (involving measuring the limb force along the limb during the limb contact on the limb contact surface) to determine both running power derived during the ground contact phase of the Gait cycle and a running economy/efficiency metric to reflect the efficiency of the user to move at any given velocity. The Gait cycle is illustrated in FIG. 4A and FIG. 4B. These measurements may be undertaken during any phase or style of limbed movement using sensors such as accelerometers or gyroscopes. For bipedal and quadrupedal motion, the Gait Cycle is defined as the time period or sequence of events during limbed motion where one limb contacts a surface to where the same limb contacts the surface again. The present invention uses two or more accelerometer measurements which allows the limb surface contact force to be derived by a mathematical algorithm, of the user during the Gait cycle. In one embodiment the present invention also uses one or more gyroscope measurements.

This is achieved through capturing and storing the outputs of two or more accelerometer sensors and in some embodiments one or more gyroscope sensors during the motion of the user. The outputs of the sensors consist of data which forms a waveform pattern which can be analysed using one or more pattern matching or in some embodiments one or more pattern recognition algorithms to detect required points in the Gait cycle for analysis. The power and running economy/efficiency metrics are then calculated using a mathematical algorithm which uses the one or more of the limb flight time, limb surface contact time, limb surface contact forces and limb velocity or centre of mass velocity measurements.

The sensors are housed in a case which may be located on any part of the limb being measured or on any part of a shoe being worn.

With reference to FIG. 1A, which shows an example embodiment of a system for determining power output and propulsion efficiency of a user's limb during limbed propulsion, the system comprises a Sensor System 100 and is configured to transmit data wirelessly to a mobile or fixed data collection and processing device 114 using a wireless protocol such as Bluetooth, Bluetooth LE, ANT or other wireless communication technology, as known in the art. The mobile or fixed data collection and processing device 114 for example, could be a mobile phone, mobile computer such as a laptop, tablet, watch, smart eyewear, or personal computer.

The Sensor System 100 is housed in a case which may be mounted on any part of the limb or, for example, on any part of footwear or a shoe attached to the limb of a user. It is to be appreciated that one or more of Sensor System 100 can be used to measure one or more of the user's limbs.

FIG. 18 illustrates an example embodiment of the Sensor System 100 and comprises of at least two accelerometers 102,103 to detect the limb's motion in orthogonal planes, in this case the vertical (X) and horizontal (Y) motion of the limb and the ground contact acceleration during the ground contact period of the Gait cycle. Without limitation, the accelerometer can be a single-axis, two-axis or three-axis device 101. The use of an accelerometer 104 to measure the third axis (Z) may be used to provide greater accuracy of the calculation of the power and efficiency metrics. It is to be appreciated that any other type of device which includes an accelerometer function, such as an inertial measurement unit (IMU) may also be used. The accelerometers are coupled to a processor 109 which receives the sensor data, stores it in memory 110, analyses the data using one or more algorithms and then calculates the power and efficiency metrics. Additional running metrics such as cadence, surface contact time and limb velocity or centre of mass velocity may also be calculated from the available data. A timer/clock function 111, provides time data used by the processor for correct operation and as inputs to the one or more algorithms implemented in the software on the Sensor System 100.

FIG. 1C illustrates another example embodiment of the Sensor System 100 and comprises at least two accelerometers 102,103 to detect the limb's motion in orthogonal planes, in this case the vertical (X) and horizontal (Y) motion of the limb and the surface/ground contact acceleration during the surface/ground contact period of the Gait cycle. Without limitation, the accelerometer can be a single-axis, two-axis or three-axis device 101. The use of an accelerometer 104 to measure the third axis (Z) may be used to provide greater accuracy of the calculation of the power and efficiency metrics. It is to be appreciated that any other type of device which includes an accelerometer function, such as an inertial measurement unit (IMU) may also be used.

The example embodiment in FIG. 1C further includes at least one or more gyroscopes which measure the orientation and angular velocity of the user and are used as inputs into the Gait detection and metric algorithms. In this embodiment, the accelerometers and gyroscope are coupled to a processor 109 which receives the sensor data, stores it in memory 110, analyses the data using one or more algorithms and then calculates the power and efficiency metrics. Additional running metrics such as cadence, limb contact time on the limb contract surface and limb velocity or centre of mass velocity may also be calculated from the available data. A timer/clock function 111, provides time data used by the processor for correct operation and as inputs to the one or more algorithms implemented in the software on the Sensor System 100.

The Sensor System 100 of the example embodiments in FIG. 19 and FIG. 1C further includes at least one wireless transmitter/receiver 113 which transmits the calculated metrics to a receiving device using a wireless protocol. In some embodiments, the metric data can be transmitted via a wired communications port 112, using an electrical data interface.

In some embodiments of the Sensor System 100, two or more of the internal components may be integrated into a single device. For example, the Processor 109, Memory 110, Timer/Clock 111, Communications Port 112 and Radio Transmitter/Receiver 113 may be integrated into a single integrated circuit.

Motion using limbs involves mechanical work. This mechanical work is performed by muscles or some form of motor which cause the limbs to accelerate and move in a direction as required by the user. Force plates have been used previously to approximately measure this external mechanical work performed by the user by recording horizontal and vertical components of surface/ground contact forces. Because of their size, force plates are not practical for use outside of a laboratory or clinical setting and mobile force sensors have limitation in ease of use for the average user. In the present invention, the surface/ground contact forces are approximated using the two or more accelerometers which form part of Sensor System 100. Despite being small, being wearable and measuring different forces (eg measuring tibial shock in the case of a runner), the accelerometer measurements provide close estimates of the metrics around power and efficiency obtained from a force plate. This is achieved by providing a measurement which allows the surface or ground contact force to be derived by a mathematical algorithm of the user during the Gait cycle. FIG. 2 shows the correlation between the outputs of a force plate and an accelerometer during the surface or ground contact phase of a typical Gait cycle of a human user. It is apparent from FIG. 2 that the plots from the accelerometer and the force plates are quite different. The accelerometer measures the ground contact acceleration, whereas the force plate measures the ground reaction force. The plots show that the accelerometer plot is much sharper and highly resolved, whereas the force plate plot is significantly more dampened and less resolved. Despite the differences, it has been found that the power and efficiency metrics obtained from the accelerometer largely approximate the metrics around power and efficiency obtained from a force plate measurement. The present invention is therefore able to provide a wearable accessory that can provide metrics around power and efficiency output.

The algorithms used to calculate power and propulsion efficiency are based on the collision mechanics that occur in any form of motion where the limb, in this illustration a leg, contacts a surface which provides a resistance force against the limb. FIG. 3 represents the point where the limb contacts with the surface during typical bipedal motion and shows the important metrics which are measured and calculated by the Sensor System 100 software. Several angles/vectors may be determined during the surface contact time of the limb comprising: the velocity angle/vector, force angle/vector and resulting collision angle. These angles/vectors are inputs to the algorithms implemented in software on the Sensor System 100 in some embodiments.

As shown in FIG. 3, the Velocity Vector 301 of the limb can be determined during the flight phase of the limb prior to surface contact. The Velocity Vector 301 during the flight phase and at surface contact comprises both magnitude and angle components which are used by some embodiments of the one or more algorithms implemented in software on the Sensor System 100. The Surface Contact Force Vector 302 at surface contact and through the surface contact phase of the Gait cycle comprises both magnitude and angle components which are used by some embodiments of the one or more algorithms implemented in software on the Sensor System 100.

In some embodiments of the one or more algorithms implemented in software on the Sensor System 100, the measured magnitude of the Velocity Vector 301 and Surface Contact Force Vector 302 are used as inputs and the angles are ignored.

The Surface Contact Force Vector 302 is measured using one or more accelerometers which measures acceleration. Illustratively the Surface Contact Force Vector 302 at any given point of the surface contact time 404 is calculated as the mass of the user times the acceleration using the equation F=ma. Force (F) is generated by the user during activity which generates motion when the user is in contact with a surface. Mass (m) is the weight of the user, which may be optionally entered into the metrics for a particular user. In some embodiments of the one or more algorithms implemented in software on the Sensor System 100, the mass of the user is normalised to a fixed numeric value. Acceleration is the acceleration measured using one or more accelerometer of the one or more points during the Surface Contact Time Vector 404.

FIGS. 4A and 4B illustrates examples of the points of a typical two limbed Gait cycle which are of interest in determining running economy/efficiency and power, according to some embodiments. FIG. 4A shows the flight phase of the Gait Cycle. The Flight Time 403 and velocity of the limb or centre of mass 301 in the horizontal plane relative to the limb contact surface from the Loss of Contact with Surface 401 at any point up to and including the Surface Contact 402 can be used as inputs to the one or more algorithms implemented in the software on the Sensor System 100. FIG. 4B shows the surface contact phase of the Gait Cycle. The Surface Contact Time 404 and Surface Contact Force 302 from Surface Contact 402 at any point up to and including Loss of Contact with Surface 401 can be used as inputs to the one or more algorithms implemented in software on the Sensor System 100.

In some embodiments of Sensor System 100 one or more algorithms using Pattern Matching techniques are implemented in software to determine the points of Loss of Contact with Surface 401 and Surface Contact 402.

In some embodiments of Sensor System 100 one or more algorithms using Pattern Recognition techniques, as used in machine learning, are implemented in software to determine the points of Loss of Contact with Surface 401 and Surface Contact 402.

In some embodiments of Sensor System 100 one or more algorithms using Pattern Matching and Pattern Recognition techniques are implemented in software to determine the points of Loss of Contact with Surface 401 and Surface Contact 402.

Power is the work done over a period of time. As distance can be derived from velocity, power can be calculated based on force and velocity. The Power metrics are calculated using the Velocity 301, Surface Contact Force 302 and Surface Contact Time 404.

The Running Economy/efficiency metric is calculated using the Velocity 301, Surface

Contact Force 302 and Surface Contact Time 404 and is a numeric value between zero and ten. A higher value denotes a user has better running economy/efficiency than a user with lower value.

In some embodiments the Running Economy/efficiency metric may be translated into an approximation of the steady state oxygen consumption as VO₂ in mL/min. This value may be adjusted according to the mass of the runner.

FIG. 5 is a flowchart illustrating an example of the software on the processor 109 and the steps performed in calculating the Power and Running Economy/efficiency metrics. Initially the Sensor System 100 is running in a low power mode at block 501 where the sensor components are either powered off or are running in a low power state to minimise current draw and extend battery life. One or more of the accelerometers are used to detect movement of the Sensor System 100 at block 502. On detection of movement the accelerometer wakes the processor 109 and the Sensor System 100 is then configured to a state with all components fully powered. At block 503 a communications link is established to a mobile or fixed data collection and processing device 114. If a communications link is not established within a fixed time period the processor 109 confirms that movement is detected at block 502 at which point it will either configure the Sensor System 100 components to operate in low power mode at block 501, if no movement is detected within a fixed time frame, or attempt to establish the communications link at block 503, if movement is detected.

Once the communications link is established to a mobile or fixed data collection and processing device 114 the processor 109 will accept configuration settings from the mobile or fixed data collection and processing device 114 at block 504 and 505. If no configuration data is provided the processor 109 starts to collect data and store it in memory 110 at block 506 using one or more of Pattern Matching or Pattern Recognition algorithms from the one or more inputs from the sensors 101, 105 that are part of the embodiment of the Sensor System 100. The one or more Pattern Matching or Pattern Recognition software algorithms that are run by the processor 109 detect the initial loss of surface contact and the initial surface contact at block 506, 507. The processor then calculates the initial limb or centre of mass Velocity 301 and flight times at block 508 and stores these in memory 110. The processor then detects the next loss of surface contact, block 509 and surface contact, block 510, then calculates the limb or centre of mass Velocity 301 and flight times at block 511, surface contact time at block 512 and Surface Contact Force 302 at block 513. The processor 109 then detects the start of the next motion cycle at block 514 and then checks that the collected data and calculations represent a valid cycle of motion before calculating the power and running economy/efficiency metrics at block 515. If the motion cycle is deemed to be invalid the data for that cycle is ignored and the processor moves to block 518 to confirm that motion is still taking place. If the motion cycle is valid the processor then calculates the power and running economy/efficiency metrics, block 516, and then transmits these using the Radio Transmitter\Receiver 113 or Communications Port 112 to the mobile or fixed data collection and processing device 114. Following this the processor checks that motion is still taking place, block 518, and the cycle continues from block 510. If motion is not detected the Sensor System 100 is configured to run in low power mode at block 501.

It can be seen from the flowchart FIG. 5 that both the limb or centre of mass Velocity 301 and flight time prior to the current Surface Contact Force 302 and the limb or centre of mass Velocity 301 and flight time post the current Surface Contact Force 302 may be used as inputs, either singularly or together as input to the Running Economy/efficiency and Power metric calculations in blocks 516, 517.

Common wireless protocols support standards or profiles for the encoding of data to enable interoperability between sensors and mobile or fixed data collection and processing device 114. The common standards however only support a small number of metrics such as heart rate, running or cycling cadence and power and there is no easy way for other metrics to be captured or displayed on common commercial implementation of mobile or fixed data collection and processing device 114. The present disclosure provides a method for encoding additional metric data in the cadence profiles used in common wireless communications protocols such as Bluetooth variants and ANT+. Instead of cadence data the Sensor System 100 can be configured to send other metrics using the cadence profile of the configured wireless standard. Metrics such as Limb or centre of mass Velocity in metres per second, Ground Contact Time in milliseconds, Tibial Acceleration in Gravities and Running Economy/efficiency data values can be transmitted instead of cadence data values. Illustratively, the cadence displayed on the mobile or fixed data collection and processing device 114 when a metric other than cadence is being encoded would represent the metric being sent. The following provide an example of how cadence metric is translated into other metrics for data capture and display. For Limb or centre of mass Velocity, a cadence of 100 is used to represent a limb or centre of mass velocity of 10.0 m/s. For Tibial Acceleration a cadence of 100 would represent 10.0 G. For Ground Contact Time a cadence of 100 would represent 100 ms. For Running Economy/efficiency a cadence of 100 would represent 10.0.

FIG. 5, blocks 504 and 505 allow for the user to provide configuration information to the Sensor System 100. This allows variables and options such as the users mass and operation of the cadence encoding function to be configured.

The example embodiments have primarily been described using bipedal lower limb motion but may also be used for all forms of quadrupedal motion such as for the measurement of equine running economy/efficiency and power.

The example embodiments may also be used for measuring the power and stroke economy/efficiency for rowing and kayaking. In this embodiment the Sensor System 100 is attached to the oar, paddle, the user's upper limb or arm.

FIG. 6 shows an example of plot of the different power metrics that might be measured in an aerobic activity versus an anaerobic activity, versus an untrained runner versus a recreational or competitive runner. Any power metrics that are provided to a user can be used as a training or coaching tool and plotted against the velocity of the runner. This also provides valuable insights into the running efficiency of the user.

### Advantages

A portable system which provides the measurement of one or both, of running power and running economy/efficiency would provide benefits to coaches, trainers and athletes who wish to measure training progress or improve Gait economy/efficiency and performance.

Accordingly, one of ordinary skill in the art will readily appreciate from the disclosure that later modifications, substitutions, and/or variations performing substantially the same function or achieving substantially the same result as embodiments described herein may be utilised according to such related embodiments of the present invention. Thus, the invention is intended to encompass, within its scope, the modifications, substitutions, and variations to processes, manufactures, compositions of matter, compounds, means, methods, and/or steps disclosed herein.

1. A system for determining power output and propulsion efficiency of a user's limb during limbed propulsion; the system comprising:
(a) at least two or more accelerometers to measure the acceleration of the limb in at least two substantially orthogonal planes,
(b) a processor means to calculate the power output generated by the limb to derive a limb propulsion efficiency measure, and
(c) a transmission means to send the limb power output and limb propulsion efficiency metrics to a receiving device; and
wherein the system is wearable on the user's limb.

2. A system for determining power output and propulsion efficiency of a user's limb during limbed propulsion; the system comprising:
(a) at least two or more accelerometers to measure the acceleration of the limb in at least two substantially orthogonal planes,
(b) at least one gyroscope to measure the rotational velocity of the limb;
(c) a processor means to calculate the power output generated by the limb to derive a limb propulsion efficiency measure, and
(d) a transmission means to send the limb power output and limb propulsion efficiency metrics to a receiving device; and
wherein the system is wearable on the user's limb.

3. The system as claimed in claim 1 having three accelerometers to measure the acceleration of the limb in three substantially orthogonal planes.

4. The system as claimed in claim 1 for measuring the power output and propulsion efficiency of the user's limb.

5. The system as claimed in claim 4, wherein the limb is the user's leg.

6. The system as claimed in claim 1 wherein the two or more accelerometers, in use, measure the limb force transmitted along the user's limb and the limb contact time on the limb contact surface and the system derives the power output and propulsion efficiency of the limb therefrom.

7. The system as claimed in claim 1 wherein the two or more accelerometers measure or are used to derive the velocity of the limb in the horizontal plane relative to the limb contact surface.

8. The system as claimed in claim 2 wherein the two or more accelerometers and one or more gyroscopes in use measure or are used to derive the velocity of the user's centre of mass in the horizontal plane relative to the limb contact surface.

9. The system as claimed in claim 1 for use on a human, robot or animal limb.

10. The system as claimed in claim 9 for use on a human limb.

11. The system as claimed in claim 9 for use on an equine limb.

12. A method for determining power output and propulsion efficiency of a user's limb during limbed propulsion; the method comprising the steps of:
(a) measuring the acceleration of the limb in at least two substantially orthogonal planes using a system, and
(b) calculating the power output generated by the limb from the acceleration measurements to derive a limb propulsion efficiency measure.

13. The method as claimed in claim 12 wherein the system as claimed claim 1 is used to measure the acceleration of the limb.

14. The method as claimed in claim 12 further including the step of transmitting the limb power output and limb propulsion efficiency metrics to a receiving device.

15. The method as claimed in claim 12 wherein the limb is a leg.

16. A method for determining power output and propulsion efficiency of a user's leg whilst running, the method including the steps of:
(a) measuring the tibial shock/force transmitted along the user's leg as it impacts the ground using a system as defined in claim 1;
(b) deriving a ground reaction force metric from the tibial shock/force measurement and the leg contact time on the ground; and
(c) measuring the acceleration in the horizontal plane of the limb or centre of mass using a system as defined in claim 1, and
(d) deriving a power output and propulsion efficiency metric therefrom.

17. The method as claimed in claim 16 further including the step of transmitting the leg power output and leg propulsion efficiency metrics to a receiving device.

18. The method as claimed in claim 11, wherein the user's limb or leg is a human, robot or animal limb or leg.

19. The method as claimed in claim 18 for use on a human leg.

20. The method as claimed in claim 18 for use on an equine leg.
